(19) United States
(12) Patent Application Publication
(10) Pub. No.: US 2024/0050721 A1
(43) Pub. Date: Feb. 15, 2024

(54) TREATMENTS FOR HEMOPHILIA A

(71) Applicant: Rani Therapeutics, LLC, San Jose, CA (US)

(72) Inventors: Mir A. IMRAN, Los Altos Hills, CA (US); Mir HASHIM, San Jose, CA (US)

(73) Assignee: Rani Therapeutics, LLC, San Jose, CA (US)

(21) Appl. No.: 18/452,358

(22) Filed: Aug. 18, 2023

Related U.S. Application Data
(63) Continuation of application No. PCT/US2022/017454, filed on Feb. 23, 2022.
(60) Provisional application No. 63/153,017, filed on Feb. 24, 2021.

(51) Int. Cl. A61M 31/00; A61K 38/37; A61M 5/315; A61K 9/48; A61P 7/04; A61K 47/60

(52) U.S. Cl. CPC A61M 31/002; A61K 38/37; A61M 5/31531; A61K 9/4808; A61P 7/04; A61K 47/60

(57) ABSTRACT

The present disclosure relates generally to treatments for hemophilia A using a swallowable device containing Factor VIII, wherein the device is structured and formulated to deliver therapeutically effective amounts of Factor VIII into the peritoneum and achieve desired pharmacokinetic and therapeutic results.

### CROSS-REFERENCE TO RELATED APPLICATION

This application claims priority under 35 U.S.C. § 119(e) to U.S. Provisional Application 63/153,017 filed Feb. 24, 2021, the entire contents of which are incorporated herein by reference.

### FIELD OF INVENTION

The present disclosure relates generally to treatments for hemophilia A using an oral composition containing Factor VIII. More specifically, the treatments use oral dosage forms (swallowable devices) as disclosed herein that are structured and formulated to deliver therapeutically effective amounts of Factor VIII into the peritoneum and achieve desired pharmacokinetic and therapeutic results.

### BACKGROUND

The following discussion is merely provided to aid the reader in understanding the disclosure and is not admitted to describe or constitute prior art thereto.

Hemophilia A is an inherited bleeding disorder in which the blood does not clot normally. People with hemophilia A will bleed more than normal after an injury, surgery, or dental procedure. This disorder can be severe, moderate, or mild. In severe cases, heavy bleeding occurs after minor injury or even when there is no injury (spontaneous bleeding). Bleeding into the joints, muscles, brain, or organs can cause pain, deformity, and other serious complications. Hemophilia A is caused by having low levels of a protein called Factor VIII, which is needed to form blood clots. Hemophilia A predominantly affects males, but can occur in females as well.

The disorder is inherited in an X-linked recessive manner and is caused by changes (mutations) in the F8 gene that encodes the Factor VIII protein. Diagnosis of hemophilia A typically is made through clinical symptoms and specific laboratory tests to measure the amount of clotting factors in the blood. The main treatment is replacement therapy, during which Factor VIII is dripped or injected slowly into a vein. Indeed, injections or infusions are the current standard of care. Due to the short half-life of Factor VIII after administration and the chronic nature of the disorder, such injections/infusions must be given frequently and for the lifetime of the patient. Moreover, patients treated with replacement therapy still may experience pathological bleeding (often referred to as “breakthrough bleeding”).

While alternative routes of administration for Factor VIII replacement therapy have been investigated, there remains a need for methods of treating hemophilia A that do not require injections or infusions.

### SUMMARY

Described herein are treatments for hemophilia A using oral dosage forms containing Factor VIII (FVIII) structured and formulated to deliver therapeutically effective amounts of FVIII into the peritoneum and achieve desired pharmacokinetic and therapeutic results.

In an aspect, the present disclosure provides methods of treating hemophilia A, including orally administering, to a subject with hemophilia A, a swallowable device containing a payload formed from, or containing, a composition including Factor VIII (FVIII), where the device is structured to deliver the FVIII into and through an intestinal wall into a peritoneal cavity of the subject. The payload may be in the form of a solid tissue penetrating member structured to penetrate an intestinal wall of the subject and/or be inserted into a peritoneal cavity of the subject after oral ingestion of the swallowable device. The swallowable device may be disposed within a capsule. The method may be effective to protect the subject from breakthrough bleeding for at least 72 hours and up to 120 hours, where the breakthrough bleeding may be spontaneous bleeding.

In an aspect, the present disclosure provides swallowable devices for use in treating hemophilia A, the devices containing a payload formed from, or containing, a composition including Factor VIII (FVIII), where the payload is in the form of a solid tissue penetrating member structured to penetrate an intestinal wall of the subject and be inserted into a peritoneal cavity of the subject after oral ingestion of the device, thereby delivering the FVIII into the peritoneal cavity of the subject. By delivery of the payload into the peritoneal cavity, the subject may be protected from breakthrough bleeding for at least 72 hours and up to 120 hours, where the breakthrough bleeding may be spontaneous bleeding.

Methods, uses and instructions associated with swallowable devices in accordance with any embodiment may include or direct administration to a subject at a frequency selected from twice per day, once per day, once every 2 days, once every 3 days, once every 4 days, once every 5 days, once every 6 days, or once every 7 days.

A kit may include one or more swallowable devices in accordance with any embodiment, and instructions for ingesting the devices.

A swallowable device in accordance with any embodiment may contain a dose of FVIII of from about 30 IU/kg to about 300 IU/kg, based on a body weight of the subject.

A swallowable device in accordance with any embodiment may contain a dose of FVIII of from about 1,000 IU to about 12,000 IU, including a dose of FVIII of 3,000 IU, 6,000 IU, or 9,000 IU. The dose of FVIII may be selected from about 75 IU/kg, about 100 IU/kg, about 125 IU/kg, about 150 IU/kg, about 175 IU/kg, about 200 IU/kg, about 225 IU/kg, about 250 IU/kg, or about 275 IU/kg. The dose of FVIII may be from about 50 IU/kg to about 250 IU/kg, from about 50 IU/kg to about 200 IU/kg, from about 50 IU/kg to about 150 IU/kg, or from about 50 IU/kg to about 100 IU/kg, based on a body weight of the subject. The dose may be less than 50 IU/kg, such as for a dose given two or more times per day.

The FVIII in a swallowable device in accordance with any embodiment may be a long-circulating form of FVIII. The FVIII may be a recombinant human PEGylated FVIII.

A swallowable device in accordance with any embodiment may be structured to provide delayed/controlled release of FVIII into the peritoneal cavity of the subject. For example, in some embodiment the device may contain multiple payloads or doses of FVIII, and may be configured to deliver the payloads or doses into the peritoneal cavity of the subject at different times. A device structured for delayed/controlled release may contain 2, 3, 4, or 5 or more payloads or doses of FVIII.

Any of the methods and uses in accordance with any embodiment may be effective in maintaining hemostasis or normalized coagulopathy in a subject for a period of time selected from at least 24 hours, at least 36 hours, at least 48 hours, at least 60 hours, at least 72 hours, at least 84 hours, or at least 96 hours, and up to 120 hours.

Any of the methods and uses in accordance with any embodiment may be effective to protect a subject from breakthrough bleeding and/or spontaneous bleeding for at least 24 hours, at least 36 hours, at least 48 hours, at least 60 hours, at least 72 hours, at least 84 hours, or at least 96 hours, and up to 120 hours.

Any of the methods and uses in accordance with any embodiment may be effective to maintain FVIII at therapeutic levels in a subject for an equal or longer period of time than intravenous administration of an equal dose of FVIII.

In specific embodiments of any of the methods or uses disclosed herein, the composition including FVIII is a dry composition filled in a hollow, biodegradable microneedle constituting the solid tissue penetrating member.

Any embodiments of a swallowable device as described herein may be biodegradable.

The foregoing general description and following detailed description are provided by way of example and are intended to provide further explanation of the disclosure as claimed, without limiting the disclosure or the claims. Other objects, advantages, and novel features will be readily apparent to those skilled in the art from the following brief description of the drawings and detailed description of the disclosure.

### BRIEF DESCRIPTION OF THE DRAWINGS

FIG. 1 (FIG. 1), FIG. 2 (FIG. 2), FIG. 3 (FIG. 3), and FIG. 4 (FIG. 4) each show an example of an embodiment of an expandable component of a self-sizing device as disclosed herein, with at least two non-compliant sections and at least one hinge.

FIG. 5A (FIG. 5A), FIG. 5B (FIG. 5B), FIG. 5C (FIG. 5C), FIG. 5D (FIG. 5D), FIG. 5E (FIG. 5E), and FIG. 5F (FIG. 5F) each show an example of an embodiment of an expandable component of a self-sizing device as disclosed herein, with at least two non-compliant sections and at least one hinge.

FIG. 6 (FIG. 6) shows an example of an embodiment of an expandable component of a self-sizing device as disclosed herein, with at least two non-compliant sections and at least two hinges.

FIG. 7A (FIG. 7A) and FIG. 7B (FIG. 7B) show an example of an embodiment of an expandable component of a self-sizing device as disclosed herein, with at least one non-compliant section and at least two hinges.

FIG. 8A (FIG. 8A) and FIG. 8B (FIG. 8B) show an example of an embodiment of an expandable component of a self-sizing device as disclosed herein, with at least two non-compliant sections and at least two hinges.

FIG. 9A (FIG. 9A) and FIG. 9B (FIG. 9B) each show an example of an embodiment of a capsule structure of a capsule as disclosed herein.

FIG. 10A (FIG. 10A) shows an example of an embodiment of an expandable component of a self-sizing device as disclosed herein prior to being folded and/or rolled, and an example of an embodiment of a capsule as disclosed herein.

FIG. 10B (FIG. 10B) shows the expandable component of FIG. 10A in an embodiment of a folded and/or rolled arrangement prior to disposing the expandable component in the capsule.

FIG. 10C (FIG. 10C) shows the expandable component of FIG. 10A in the folded and/or rolled arrangement of FIG. 10B and disposed within the capsule.

FIG. 11A (FIG. 11A), FIG. 11B (FIG. 11B), and FIG. 11C (FIG. 11C) show an example of an embodiment of a swallowable capsule including a self-sizing device as disclosed herein, including an expandable component within a degradable capsule, as the device traverses a lumen.

FIG. 11D (FIG. 11D), FIG. 11E (FIG. 11E), and FIG. 11F (FIG. 11F) show a progression of the expandable component of FIG. 11C in a rotated view as the expandable component expands within the lumen.

FIG. 12 (FIG. 12) shows an example of an embodiment of a self-sizing device as disclosed herein including an expandable component in a fully-extended state within a lumen.

FIG. 13A (FIG. 13A) shows an example of an embodiment of a self-sizing device as disclosed herein including an expandable component with one non-compliant section and no hinge.

FIG. 13B (FIG. 13B) shows an example of an embodiment of a self-sizing device including an expandable component with two non-compliant sections and one hinge.

FIG. 14 (FIG. 14) shows activated partial thromboplastin time (aPTT), following an intraperitoneally-injected (IP-injected) administration of FVIII versus a capsule-delivered administration of FVIII as disclosed herein.

FIG. 15 (FIG. 15) shows FVIII activity levels, following an IP-injected administration of FVIII versus a capsule-delivered administration of FVIII as disclosed herein.

FIG. 16 (FIG. 16) and FIG. 17 (FIG. 17) show comparisons of data for the IP-injected dose versus the capsule-delivered dose, specifically WBCT plotted against FVIII Activity (FIG. 16) and aPTT plotted against FVIII Activity (FIG. 17).

FIG. 18 (FIG. 18) shows an embodiment of a capsule as disclosed herein encompassing a swallowable device. Inset A shows a fully assembled enteric-coated capsule. Schematic B shows various parts and components of the capsule.

### DETAILED DESCRIPTION

People with hemophilia A face a lifelong burden of frequent intravenous FVIII injections. The convenience of an oral FVIII therapy would greatly improve compliance and quality of life for these patients.

The present disclosure provides methods of treating hemophilia A using a swallowable device containing a payload formed from, or containing, a composition including Factor VIII (FVIII), where the device is structured to deliver the FVIII into and through an intestinal wall or into a peritoneal cavity of the subject. The payload may be in the form of a solid tissue penetrating member structured to penetrate an intestinal wall of the subject and/or be inserted into a peritoneal cavity of the subject after oral ingestion of the swallowable device. The swallowable device may be disposed within a capsule.

### I. Definitions

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more”.

As used herein, the terms “e.g.,” “such as”, “for example”, “for an example”, “for another example”, “examples of”, “by way of example”, and “etc.” indicate that a list of one or more non-limiting example(s) precedes or follows; it is to be understood that other examples not listed are also within the scope of the present disclosure.

As used herein, the terms “substantially” and “about” when used with a numerical value mean the numerical value stated as well as up to and including plus or minus 10% of the numerical value. For example, “about 10” should be understood as both “10” and “in a range between and including 9 and 11”.

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the terms “comprising”, “comprise”, “comprises”, “includes”, and “including” are intended to mean that the compositions and methods include the recited elements, but do not exclude others.

As used herein, the phrase “therapeutically effective amount” with reference to FVIII means a dose of FVIII that provides the specific pharmacological effect for which the drug is administered in a subject in need of such treatment. A therapeutically effective amount may be effective to reduce the risk of, reduce, ameliorate, or eliminate bleeding episodes or events (e.g., breakthrough bleeding or spontaneous bleeding) and/or improve clotting function (e.g., achieve hemostasis or normalized coagulopathy) in a subject with hemophilia A. It is emphasized that a therapeutically effective amount of FVIII will not always be effective in treating hemophilia A in every individual subject, even though such dose is deemed to be a therapeutically effective amount by those of skill in the art. Those skilled in the art can adjust what is deemed to be a therapeutically effective amount in accordance with standard practices as needed to treat a specific subject. A therapeutically effective amount may vary based on, for example, the age and weight of the subject, and/or the subject's overall health, and/or the severity of the condition of the subject being treated. A therapeutically effective amount may prevent bleeding episodes or events, such as breakthrough bleeding.

The term “therapeutic level” when used in reference to FVIII and the treatment of hemophilia A means an amount of circulating FVIII in a subject that is sufficient to provide normalized coagulopathy or hemostasis and/or reduce the risk or rate of occurrence of breakthrough bleeding.

The term “breakthrough bleeding” refers to bleeding that occurs despite prophylactic treatment with FVIII or another medication for treating hemophilia A. “Spontaneous bleeding” is a type of breakthrough bleeding that occurs in the absence of any injury or apparent cause.

The term “hemostasis” refers to a physiological process of slowing and stopping a flow of blood, such as stopping a flow of blood through a blood vessel wall, stopping of bleeding, or stopping of a bleed.

The term “normalized coagulopathy” refers to an acceptable clotting time for a hemophilic subject as measured by whole blood clotting time (WBCT). It should be noted that “normalized coagulopathy” may be distinct from what a hematologist could describe as “normalized coagulation”. For example, non-hemophiliac humans may have a “normal” WBCT of 8-12 minutes, whereas a WBCT of 20 minutes for a hemophiliac may be considered “normalized coagulation” by a person skilled in the art.

The terms “individual,” “subject,” and “patient” are used interchangeably herein, and refer to any individual animalian subject (e.g., bovine, canine, feline, equine, or human). In specific embodiments, the subject, individual, or patient is a human.

The term “component” refers herein to one item of a set of one or more items that together make up a device, a composition, or a system under discussion. A component may be in a solid, powder, gel, plasma, fluid, gas, or other constitution. For example, a device may include multiple solid components which are assembled together to structure the device and may further include a fluid component that is disposed in the device. For another example, a composition may include a single component, or two or more components which are mixed together to make the composition. A composition may be in the form of a fluid, a slurry, a powder, or a solid (e.g., in a condensed or a consolidated form such as a tablet or microtablet). A device or system can include one or more compositions and/or one or more other components.

The term “design” or a grammatical variation thereof (e.g., “designing” or “designed”) refers herein to characteristics intentionally incorporated based on, for example, estimates of tolerances (e.g., component tolerances and/or manufacturing tolerances) and estimates of environmental conditions expected to be encountered (e.g., temperature, humidity, external or internal ambient pressure, external or internal mechanical pressure, stress from external or internal mechanical pressure, age of product, or shelf life, or, if introduced into a body, physiology, body chemistry, biological composition of fluids or tissue, chemical composition of fluids or tissue, pH, species, diet, health, gender, age, ancestry, disease, or tissue damage); it is to be understood that actual tolerances and environmental conditions before and/or after delivery can affect characteristics so that different components, devices, compositions, or systems with a same design can have different actual values with respect to those characteristics. Design encompasses also variations or modifications before or after manufacture.

The term “manufacture” or a grammatical variation thereof (e.g., “manufacturing” or “manufactured”) as related to a component, device, composition, or system refers herein to making or assembling the component, device, composition, or system. Manufacture may be wholly or in part by hand and/or wholly or in part in an automated fashion.

The term “structured” or a grammatical variation thereof (e.g., “structure” or “structuring”) refers herein to a component, device, composition, or system that is manufactured according to a concept or design or variations thereof or modifications thereto (whether such variations or modifications occur before, during, or after manufacture) whether or not such concept or design is captured in a writing.

The term “degrade” or a grammatical variation thereof (e.g., “degrading”, “degraded”, “degradable”, and “degradation”) refers herein to weakening, partially degrading, or fully degrading, such as by dissolution, chemical degradation (including biodegradation), decomposition, chemical modification, mechanical degradation, or disintegration, which encompasses also, without limitation, dissolving, crumbling, deforming, shriveling, or shrinking.

The term “non-degradable” refers to an expectation that degradation will be minimal, or within a certain acceptable design percentage, for at least an expected duration in an expected environment.

The terms “FVIII composition” and “composition of FVIII” may be used interchangeably herein and refer to a composition including one or more components where at least one of the components is FVIII. The FVIII composition can include, for example, FVIII either as the sole active agent or including one or more additional active agents.

The term “FVIII” may refer to either FVIII derived from human plasma or recombinant FVIII.

As used herein, a “delay agent” refers to a component included in the composition to slow a release rate of one or more other component(s) from a composition. A delay agent may be, for example, poly(lactic acid) (PLA), poly(glycolic acid) (PGA), polyethylene glycol (PEG), poly(ethylene oxide) (PEO), poly (I-lactic acid) (PLLA), poly(D-lactic acid) (PDLA), another polymer, or a hydrogel.

As used herein, a “long-circulating form” or “long-acting form” of FVIII refers to a form that has been modified with a delay agent that prolongs the in vivo half-life (e.g., circulation) of the FVIII. A “long-circulating form of FVIII” may include FVIII conjugated to any of PEG, PLA, PGA, PEO, PLLA, PDLA, or another polymer, hydrogel, or other delay agent.

Various abbreviations may be used herein for standard units, such as deciliter (dl), milliliter (ml), microliter (μl), international unit (IU), centimeter (cm), millimeter (mm), nanometer (nm), inch (in), kilogram (kg), gram (gm), milligram (mg), microgram (μg), nanogram (ng), millimole (mM), degrees Celsius (° C.), degrees Fahrenheit (° F.), millitorr (mTorr), hour (hr), minute (min), second (s or sec), millisecond (ms), microsecond (μs), or nanosecond (ns).

### II. Hemophilia A

Hemophilia A, also called Factor VIII (factor 8) deficiency or classic hemophilia, is a genetic disorder caused by missing or defective FVIII, a clotting protein. Although it is passed down from parents to children, about ⅓ of cases found have no previous family history. Nevertheless, Hemophilia A is an inheritable disease. The gene for hemophilia is carried on the X chromosome, and therefore hemophilia A is inherited in an X-linked recessive manner.

Approximately 60% of cases of hemophilia A are considered severe, which means the subject's FVIII levels are less than 1%. Approximately 15% of cases are considered moderate (FVIII levels of 1-5%) and approximately 25% of cases are considered mild (FVIII levels of 5%-30%). A “normal” range of FVIII is, for example, levels greater than 50%. The technology disclosed herein can treat mild, moderate, and/or severe hemophilia A.

People with hemophilia A bleed longer than other people. Bleeds can occur internally, into joints and muscles, or externally, from minor cuts, dental procedures, or injuries. People with mild hemophilia A generally experience bleeding typically only after serious injury, trauma, or surgery. In many cases, mild hemophilia is not diagnosed until an injury, surgery or tooth extraction results in prolonged bleeding. The first episode may not occur until adulthood. Women with mild hemophilia often experience heavy menstrual bleeding, and can hemorrhage (bleed extensively) after childbirth. People with moderate hemophilia A tend to have bleeding episodes after injuries and may occasionally experience spontaneous bleeding. People with severe hemophilia A experience bleeding following an injury and may have frequent spontaneous bleeding episodes—bleeds that occur without obvious cause—often into their joints and muscles.

Most conventional treatments for hemophilia A focus on replacing the missing protein, FVIII, so a person can form a clot, and so reduce or eliminate the bleeds associated with the disorder. Thus, the main medication to treat hemophilia A is a concentrated FVIII product. This protein-replacement therapy is generally injected or infused into a vein in the arm or hand, or through a port in the chest. To maintain enough clotting factor in the bloodstream to prevent bleeds, patients with severe hemophilia (or moderate hemophilia, and possibly mild hemophilia) are typically prescribed a regular treatment regimen, called prophylaxis. This means a person will infuse their medication on a regular schedule; for example, multiple times per day, every day, every other day, or once every three days, depending on how long the FVIII lasts in the body. This type of prophylaxis is currently the standard of care for treating hemophilia A in subjects in need of treatment, and it is considered to be optimal therapy for most people with severe hemophilia A. Despite the current standard of care of injections and infusions, many subjects will still experience breakthrough bleeding, which occurs spontaneously or as a result of injury, even though the subject is receiving prophylaxis. Indeed, the prevention and length of time of controlling breakthrough bleeding is considered a measure of success in treating hemophilia A, and current therapies leave significant room for improvement.

### III. Methods of Treatment and Pharmaceutical Uses

The current standard of care for treating hemophilia A requires lifelong, regular injections and/or infusions of FVIII to replace the missing or defective protein. Moreover, even the current standard of FVIII replacement therapy (i.e., prophylaxis) cannot prevent breakthrough bleeding for extended periods of time. The present disclosure addresses this limitation by providing an oral route of administration that effectively delivers FVIII into the peritoneal cavity of a subject being treated and prevent breakthrough bleeding for extended periods of time. The disclosed doses, dosing schedules, and regimens provide for effective prophylactic treatment and control of hemophilia A, as demonstrated by hemostasis and/or normalized coagulopathy, and the disclosed treatments are more convenient and less invasive than the current standard or care.

The present disclosure provides methods of treating hemophilia A with an oral dosage form (e.g., a swallowable device) containing FVIII. The present disclosure also provides methods of protecting a subject with hemophilia A from breakthrough bleeding (e.g., reducing the risk of breakthrough bleeding) with an oral dosage form containing FVIII. The present disclosure provides uses of the described oral dosage forms containing FVIII for treating hemophilia A and/or protecting a subject with hemophilia A from breakthrough bleeding.

The dosage forms used in the methods described herein can be swallowable devices (i.e., oral dosage forms) structured to deliver Factor VIII into and through an intestinal wall or into a peritoneal cavity of a subject with hemophilia A. In general, the devices contain a payload that is formed from or contains a composition including FVIII. The payload may be shaped as a solid tissue penetrating member structured to penetrate an intestinal wall of the subject and be inserted into and through an intestinal wall or into a peritoneal cavity of the subject after oral ingestion of the device, thereby delivering the FVIII into the peritoneal cavity of the subject. The payload may be formed from, or may contain, the composition. The devices may be encompassed within a biodegradable capsule. The devices may be biodegradable. Specific embodiments of such devices are described in more detail below. Other embodiments of such devices suitable for use in practicing the methods and uses disclosed herein are disclosed in U.S. Pre-Grant Publication 2019/0133937 and U.S. Pre-Grant Publication 2020/0222318 the entire contents of which are incorporated herein by reference in their entirety.

Thus, this disclosure provides a FVIII-containing capsule/device of any of the embodiments disclosed herein for use in treating hemophilia A and/or protecting a subject with hemophilia A from breakthrough bleeding and/or spontaneous bleeding. This disclosure also provides uses of any of the foregoing embodiments of a FVIII-containing capsule/device of any of the embodiments disclosed herein for treating hemophilia A and/or protecting a subject with hemophilia A from breakthrough bleeding and/or spontaneous bleeding.

The following section provides more details with respect to the FVIII composition, dosing, dosing regimens/schedules, clinical endpoints, and further embodiments of the disclosed methods and uses.

More specifically, the present disclosure provides methods of treating hemophilia A, including orally administering, to a subject with hemophilia A, a swallowable device containing a payload formed from or comprising a composition including FVIII. The payload may be in the form of a solid tissue penetrating member structured to penetrate an intestinal wall of the subject and be inserted into a peritoneal cavity of the subject after oral ingestion of the device. The oral administration of such swallowable devices may be repeated, for example, at a frequency of once every 1-3 days or less frequently. The payload may be formed from, or may contain, the composition including FVIII. The payload is protected from the gastrointestinal (GI) tract environment (e.g., protected against degradation due to ingress of fluid reaching the payload) until the payload is injected into the wall of the small intestine, which is insensitive to sharp stimuli so that the subject does not register pain from the injection.

The present disclosure also provides methods of treating hemophilia A, including orally administering, to a subject with hemophilia A, a swallowable device containing a payload including a composition including FVIII. The payload may be in the form of a solid tissue penetrating member structured to penetrate an intestinal wall of the subject and be inserted into a peritoneal cavity of the subject after oral ingestion of the device, thereby delivering the FVIII into the peritoneal cavity of the subject. The payload may be formed from, or may contain, the composition. In an embodiment, the device contains a dose of FVIII of from about 30 IU/kg to about 300 IU/kg, based on a body weight of the subject.

The present disclosure also provides methods of protecting a subject with hemophilia A from breakthrough bleeding, including orally administering, to a subject with hemophilia A, a swallowable device containing a payload including a composition including FVIII. The payload may be in the form of a solid tissue penetrating member structured to penetrate an intestinal wall of the subject and be inserted into a peritoneal cavity of the subject after oral ingestion of the device, thereby delivering the FVIII into the peritoneal cavity of the subject. The payload may be formed from, or may contain, the composition. The method may be effective to protect the subject from breakthrough bleeding for at least 72 hours and up to 120 hours, wherein the breakthrough bleeding may be spontaneous bleeding.

The present disclosure also provides a swallowable device containing a composition including FVIII for use in treating hemophilia A or protecting a subject with hemophilia A from breakthrough bleeding. The payload may be in the form of a solid tissue penetrating member structured to penetrate an intestinal wall of the subject and be inserted into a peritoneal cavity of the subject after oral ingestion of the device, thereby delivering the FVIII into the peritoneal cavity of the subject. The payload may be formed from, or may contain, the composition. The breakthrough bleeding may be spontaneous bleeding. The device may be administered at a frequency of every 1-3 days or less frequently. The device may contain a dose of FVIII of from about 30 IU/kg to about 300 IU/kg, based on a body weight of the subject. As a result of the treatment, the subject may be protected from breakthrough bleeding for at least 72 hours and up to 120 hours.

The disclosed methods and uses provide a significant improvement over current standard of care treatments for hemophilia A for multiple reasons. For instance, there is not currently an orally administered treatment for hemophilia A; rather, current treatments are administered via injection. Therefore, the methods and uses of the present disclosure can fill an unmet need. Additionally, the disclosed methods and uses can allow for more convenient dosing regimens or schedules. The examples herein show that a single administration of a device as disclosed herein can provide a subject with protection from breakthrough bleeding, spontaneous bleeding events, and general protections from the symptoms of hemophilia A for 3, 4, or 5 or more days. Nevertheless, for convenience, the devices may be administered once a day so as to establish a daily routine for simplicity to improve compliance. Importantly, overdosing of FVIII is not generally a concern, so that even higher doses could be delivered daily. Thus, unless otherwise specified, the disclosed methods and uses encompass daily administration (once per day). However, the disclosed methods and uses also encompass administration multiple times per day, every other day, once every 3 days, once every 4 days, once every 5 days, once every 6 days, or once every 7 days (once per week).

The disclosed methods and uses encompass FVIII compositions that include a long-acting form of FVIII (e.g., PEGylated FVIII), and further encompass delayed-release payloads that are structured to release a first dose of a FVIII composition contained within the payload at one time and another dose of a FVIII composition contained within the payload at a later time. For example, a delayed-release payload may include multiple FVIII compositions, such as multiple instances of one FVIII composition (either long-acting or not) or multiple different FVIII compositions (e.g., one or more long-acting FVIII compositions and one or more FVIII compositions that are not long-acting), where each of the multiple FVIII compositions is released from the delayed-release payload at a different time. Examples of suitable delayed/controlled release payloads and devices containing them are disclosed in PCT/US2020/054559, the entire contents of which are incorporated herein by reference in their entirety. Such delayed-release payload containing multiple FVIII compositions released at different times can increase a duration of a period between ingestions of devices including delayed-release payloads, by days or weeks as compared to single-dose payloads. Alternatively, the device can be configured to deliver a payload or dose out of the multiple payloads or doses frequently, such as to simulate a continuous dosing regimen. For any of these embodiments, the device may contain multiple payloads or doses of FVIII, and the device can be structured to deliver the payloads or doses into the peritoneal cavity of the subject at different times. A device structured for delayed/controlled release may contain 2, 3, 4, or 5 or more payloads or doses of FVIII.

The disclosed methods and uses also provide a benefit of requiring a lower dose than conventional injections or infusions. Thus, for use in the disclosed methods and uses, the device may contain a dose of FVIII of from about 1,000 IU to about 12,000 IU. For example, the dose may be about 1,000 IU, about 1,500 IU, about 2,000 IU, about 2,500 IU, about 3,000 IU, about 3,500 IU, about 4,000 IU, about 4,500 IU, about 5,000 IU, about 5,500 IU, about 6,000 IU, about 6,500 IU, about 7,000 IU, about 7,500 IU, about 8,000 IU, about 8,500 IU, about 9,000 IU, about 9,500 IU, about 10,000 IU, about 10,500 IU, about 11,000 IU, about 11,500 IU, or about 12,000 IU. In specific embodiments the device may contain a dose of FVIII of 3,000 IU, 6,000 IU, or 9,000 IU.

Doses suitable for use in the method and uses disclosed herein may be based on the weight of the subject being treated. For example, the dose of FVIII may be selected from about 75 IU/kg, about 80 IU/kg, about 85 IU/kg, about 90 IU/kg, about 95 IU/kg, about 100 IU/kg, about 105 IU/kg, about 110 IU/kg, about 115 IU/kg, about 120 IU/kg, about 125 IU/kg, about 130 IU/kg, about 135 IU/kg, about 140 IU/kg, about 145 IU/kg, about 150 IU/kg, about 155 IU/kg, about 160 IU/kg, about 165 IU/kg, about 170 IU/kg, about 175 IU/kg, about 180 IU/kg, about 185 IU/kg, about 190 IU/kg, about 195 IU/kg, about 200 IU/kg, about 205 IU/kg, about 210 IU/kg, about 215 IU/kg, about 220 IU/kg, about 225 IU/kg, about 230 IU/kg, about 235 IU/kg, about 240 IU/kg, about 245 IU/kg, about 250 IU/kg, about 255 IU/kg, about 260 IU/kg, about 265 IU/kg, about 270 IU/kg, or about 275 IU/kg. In specific embodiments, the dose of FVIII may be from about 50 IU/kg to about 250 IU/kg, from about 50 IU/kg to about 200 IU/kg, from about 50 IU/kg to about 150 IU/kg, or from about 50 IU/kg to about 100 IU/kg, based on a body weight of the subject. In further specific embodiments, the dose of FVIII may be from 50 IU/kg to 150 IU/kg. When dosing is based on the body weight of the subject, the present disclosure contemplates patients of all different body weights, such as 20-150 kg. For example, a non-obese adult subject may be 50-90 kg or 60-80 kg. An obese adult subject may be more in the range of 100-150 kg. Pediatric subjects (e.g., subjects that are less than 18 years of age) may be 20-60 kg.

In some embodiments, the composition including FVIII is a dry composition filled inside a hollow, biodegradable microneedle constituting a solid tissue penetrating member. However, the form of the composition including FVIII is not particularly limited. For example, in some embodiments, the composition including FVIII may be a dry composition that is formed into a biodegradable microneedle constituting the solid tissue penetrating member. Such embodiments are described in more detail in U.S. Pre-Grant Publication 2019/0133937 and U.S. Pre-Grant Publication 2020/0222318, the entire contents of which are incorporated herein by reference in their entirety.

Any form of FVIII suitable for use in the subject being treated may be used. As noted above, the FVIII may be a long-circulating (long-acting) form of FVIII, such as PEGylated FVIII. Other moieties may be used to increase the half-life of the FVIII. For example, long-circulating forms of FVIII may include FVIII conjugated to PLA, PGA, PEG, PEO, PLLA, PDLA, or another polymer, or a hydrogel. Additionally or alternatively, the FVIII may be a recombinant FVIII, such as a recombinant human FVIII. The FVIII may be a recombinant human PEGylated FVIII. The FVIII may be derived from human blood plasma.

The disclosed methods and uses have been surprisingly shown to maintain hemostasis and protect against breakthrough bleeding and/or spontaneous bleeding for extended amounts of time following a single administration of a swallowable device as disclosed herein. For example, treated subjects may maintain hemostasis or normalized coagulopathy after a single administration as disclosed herein for a period of time selected from at least 24 hours, at least 36 hours, at least 48 hours, at least 60 hours, at least 72 hours, at least 84 hours, or at least 96 hours, and up to 120 hours or longer. Additionally or alternatively, a treated subject may be protected from breakthrough bleeding and/or spontaneous bleeding for at least 24 hours, at least 36 hours, at least 48 hours, at least 60 hours, at least 72 hours, at least 84 hours, or at least 96 hours, and up to 120 hours or longer after a single administration as disclosed herein.

Also surprisingly, the disclosed methods and uses may maintain FVIII at therapeutic levels in the subject for an equal or longer period of time than intravenous administration of an equal dose of FVIII. For instance, the disclosed methods and uses may maintain FVIII at therapeutic levels in the subject for at least 24 hours, at least 36 hours, at least 48 hours, at least 60 hours, at least 72 hours, at least 84 hours, or at least 96 hours, and/or up to 120 hours or longer, after a single administration as disclosed herein.

The disclosed methods and uses may include a treatment regimen (e.g., dosing schedule) that maintains FVIII exposure sufficient to reduce a risk of or prevent breakthrough bleeding. For example, breakthrough bleeding may be prevented for at least 72 hours and up to 120 hours or more, after a single administration as disclosed herein. For example, a treated subject may not experience breakthrough bleeding for at least 72 hours, at least 78 hours, at least 84 hours, at least 90 hours, at least 96 hours, at least 102 hours, at least 108 hours, at least 114 hours, 120 hours or more, after a single administration as disclosed herein.

### IV. Oral Dosage Forms for Delivering FVIII

In general, the disclosed methods and uses are contemplated for treating hemophilia A in a subject. In human subjects, the age and size of the human is not limited, and may include pediatric, adult, and geriatric subjects, as well as subjects at, above, or below a normal range of body weight or height.

As noted above, the dosage forms used in the methods described herein are swallowable devices that contain a payload made from or comprising a composition including FVIII. The payload may be in the form of a solid tissue penetrating member structured to penetrate an intestinal wall of the subject and be inserted into a peritoneal cavity of the subject after oral ingestion of the device, thereby delivering the FVIII into the intestinal wall or the peritoneal cavity of the subject. The payload may be formed from, or may contain, the composition. Specific embodiments of such devices are described in more detail below. Other embodiments of such devices suitable for use in practicing the methods and uses disclosed herein are disclosed in U.S. Pre-Grant Publication 2019/0133937 and U.S. Pre-Grant Publication 2020/0222318, the entire contents of which are incorporated herein by reference in their entirety. Any devices disclosed or referenced herein optionally may be disposed within a capsule. Typically, the materials used to form the components of the devices are classified as food grade, food additive, active or inactive food ingredient or GRAS (Generally Recognized As Safe by the FDA). The devices (including any capsule shell) may be biodegradable.

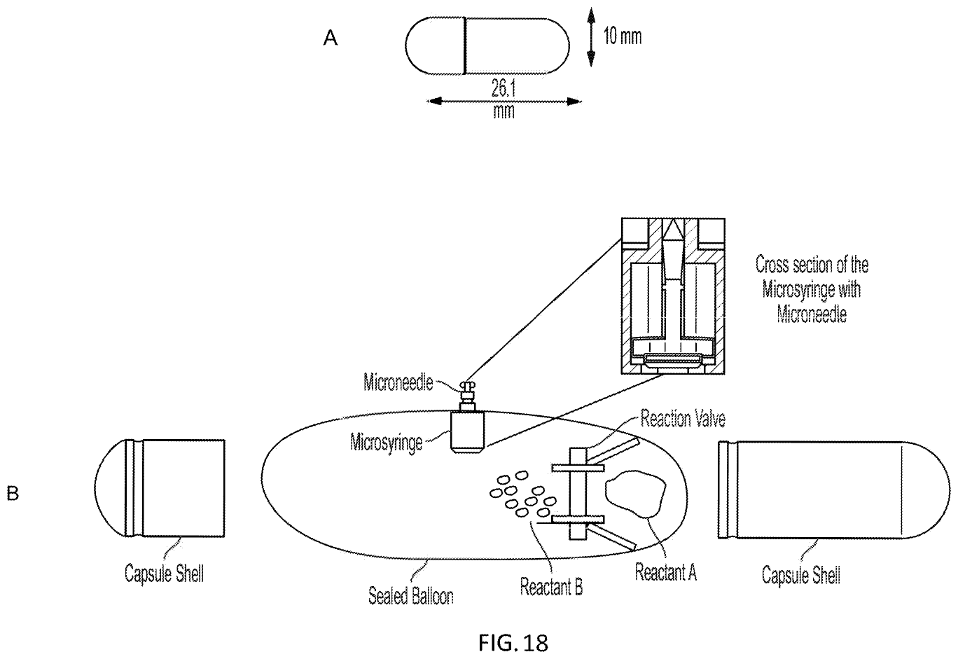

FIG. 18 diagrammatically illustrates a swallowable device according to an embodiment of this disclosure. As illustrated, the device is disposed within a capsule, which may be any suitably sized swallowable capsule, such as a 000 sized HPMC (hydroxypropyl methylcellulose) capsule, or such as the capsule illustrated in inset A of FIG. 18. The capsule may be provided with an enteric coating, such as an enteric polymer, (e.g., a polymer that dissolves at a target pH within the digestive tract). For example, the enteric coating can be selected to dissolve at a pH >6 to avoid dissolution in the acidic environment of the stomach.

In those embodiments in which the device is incorporated into an enteric coated capsule, after the capsule leaves the stomach and enters the duodenum, the higher intestinal pH of the small intestine dissolves the enteric coating and capsule shell, exposing an interior of the device and the components contained therein to intestinal fluid.

The device may contain a mechanism to effectuate delivery of the payload into the intestinal wall. In an embodiment, the mechanism includes an actuator that is triggered when the device is at a target delivery site, such as within the small intestine.

In the embodiment of FIG. 18, the device contains a sealed polyethylene balloon (labeled Sealed Balloon), a delivery construct (labeled Microsyringe), and an actuator in the form of two reactants and a valve, all of which together constitute a mechanism to effectuate delivery of the payload into the intestinal wall. In this embodiment, the Microsyringe contains the payload (labeled Microneedle). The two reactants, labeled Reactant A and Reactant B (e.g., citric acid and potassium bicarbonate), are separated by a valve (labeled Reaction Valve) in an initial state. When exposed to fluid such as intestinal fluid (e.g., after the capsule degrades), a portion of the valve degrades and allows the two reactants to mix, which results in formation of a gas (e.g., carbon dioxide or other innocuous gas) that expands the balloon. Inflation of the balloon aligns the Microsyringe perpendicular to the long axis of the intestine and builds pressure in the balloon sufficient to provide the force needed to eject the Microneedle from the Microsyringe and inject the Microneedle into and through the intestinal wall. After microneedle deployment, the balloon deflates and the balloon and other components are excreted out through the GI tract with normal bowel movements.

In general, the mechanism to effectuate delivery of the payload into the intestinal wall may include various mechanical, electrical, electromechanical, and/or chemical components. For example, springs, levers, and/or various movable components may together constitute the mechanism.

The payload may be disposed within a protective enclosure until just before (e.g., in terms of ms or las) the payload is delivered into the intestinal wall. For example, in the embodiment of FIG. 18, the Microneedle may be sealed within the Microsyringe in the initial state, and when ejected from the Microsyringe into the intestinal wall, may pierce or break a seal on the Microsyringe to pass out of the Microsyringe.

After passing through the intestinal wall and into a peritoneal cavity, the payload dissolves or degrades in the moist tissue environment to release the FVIII composition into vasculature within the peritoneal cavity. The payload may be a hollow device in which the FVIII composition is disposed, or the payload is itself formed from the FVIII composition (e.g., the FVIII composition may be compressed into a desired size and shape). In an embodiment, the FVIII composition is compressed into a cylindrically-shaped microtablet or other shaped microtablet. In an embodiment, the FVIII composition in uncompressed or compressed form is disposed within a degradable shell, and the shell containing the microtablet is the payload. In an embodiment, the shell is approximately needle-shaped. Degradation of the payload may occur within 1, 2, 3, 4, or 5 minutes of injection into the intestinal wall, or longer. Properties of the payload (e.g., structure or material) may delay release of the FVIII from the payload.

The device may include a detectable marker, such as a radiolabel, to assist in tracking the device as it proceeds through the subject's GI tract. Examples of markers include, but are not limited to, barium sulfate (whose dispersion indicates fluid ingress inside the device and imminent delivery of the payload) and bismuth, which optionally may be included on or in a component contained in the device to radiographically track the component's transit along the GI tract as well as to confirm its excretion.

In an embodiment, which may be similar to the embodiment illustrated in FIG. 18, a balloon is self-sizing. With respect to the embodiment including the self-sizing balloon, the balloon is referred to herein as an “expandable component”.

In some embodiments, the expandable component includes multiple sections. In the expanded configuration of the expandable component, at least one of the sections is non-compliant and at least one of the sections is compliant. Compliant refers to a state in which the section may be readily deformed, and non-compliant refers to a state in which the section resists deformation.

Absent a constraint, each section will expand to a fully-extended state and the expandable component will reach its maximum dimensions, which may depend, for example, on materials used to form the expandable component and/or a capacity of an expansion module that is implemented (e.g., for expansion by inflation, maximum dimensions may be influenced by a limitation on an inflation force available from the expansion module, or by a stretch factor of a material or materials of the expandable component). The expansion module is part of a mechanism to effectuate delivery of the payload into the intestinal wall.

Each compliant section operates in similar fashion to a hinge and thus will be referred to for convenience as a hinge. In a fully-extended state of the expandable component (where each section is fully extended), each hinge has at least one design dimension (width, length, and/or circumference) that is substantially smaller than a corresponding design dimension of each non-compliant section.

As the expandable component expands, it will achieve a shape that reflects an equilibrium between a force exerted by the expandable component on an interior of a lumen of the GI tract and a force of the lumen against the expandable component. Said another way, the expandable component may not fully extend and will tend to be bent about the hinge if constrained by the lumen. This bending is due to the smaller dimension(s) of the hinge as compared to the corresponding dimension(s) of the non-compliant section(s), which in the expanded configuration of the expandable component leads to lower rigidity of the hinge as compared to rigidity of the non-compliant section(s).

In this way, the expandable component expands until the non-compliant sections (which may hereinafter be referred to as NCS) are pressed against the lumen inner wall to maintain the expandable component in a position suitable for delivering a FVIII composition to the lumen wall, for a time at least sufficient to accomplish such delivery. The expandable component may then be deflated.

The expandable component may be removed after deflation, may be left to degrade in situ, or may be allowed to pass out of the lumen. The expandable component can be structured from degradable materials such that the expandable component degrades after a designed period of time after exposure to an environment at a target site.

In some embodiments, the self-sizing balloon device is particularly suited to avoid delivery being affected by matter present in a lumen. For example, the self-sizing balloon device can have a capability by rapid expansion of the expandable component to push matter out of the way and/or to compress matter against the lumen wall, expanding to the extent appropriate for a desired delivery rate for the delivery technique and the composition of FVIII to be delivered without further expansion. For a self-sizing balloon device structured for delivery into a GI lumen, the self-sizing balloon device can accomplish such delivery whether the subject is in a fed or fasted state (meaning, that delivery is accomplished whether there is digestive matter in the GI lumen or not).

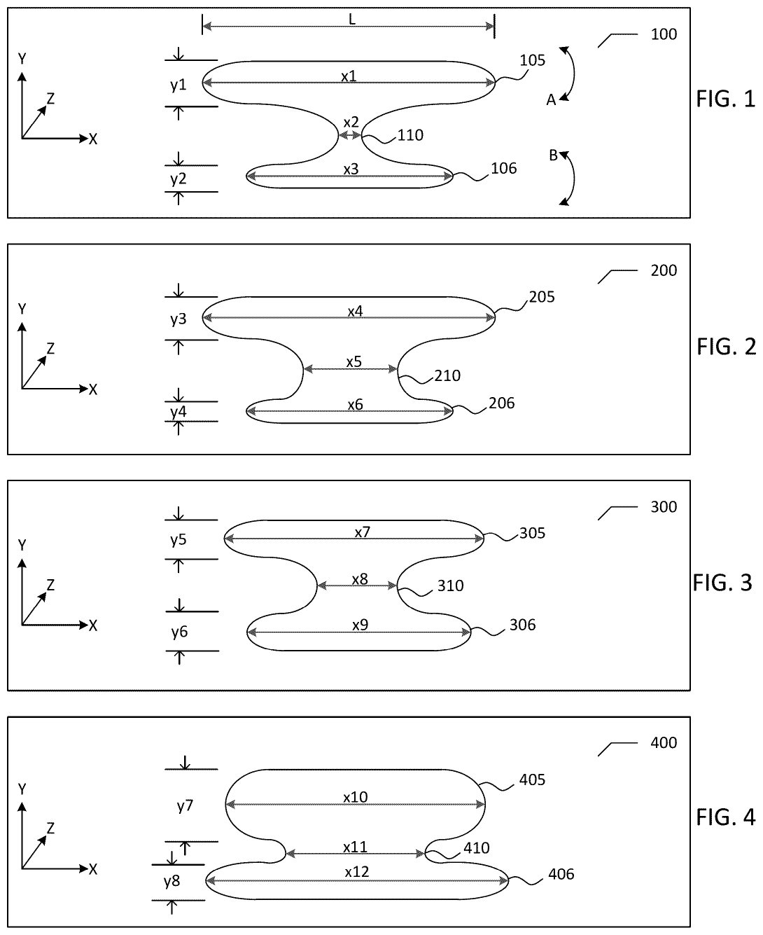

FIGS. 1-4 illustrate examples of embodiments of expandable components in fully-extended states in which the expandable components are extended without constraint to maximum dimensions (which may depend, for example, on material(s) used for the expandable components and a capability of an expansion module, among other considerations). An arbitrarily assigned x-y-z frame of reference is indicated for convenience of discussion, and the expandable components of FIGS. 1-4 are shown in an x-y reference plane. Each expandable component includes two NCSs and one hinge in the x-y plane shown.

Referring to FIG. 1, an expandable component 100 includes an NCS 105 and an NCS 106 separated by a hinge 110. In this view of this embodiment as illustrated in FIG. 1A, a dimension x1 of NCS 105 is greater than a dimension x3 of NCS 106, and both x1 and x3 are greater than a dimension x2 of hinge 110. Said another way, x1/x3>1, x1/x2>1, and x3/x2>1. Further in this view of this embodiment, a dimension y1 of NCS 105 is greater than a dimension y2 of NCS 106. In this view, then, NCS 105 is taller and longer than NCS 106.

Often, the elongated shape of an expandable component (e.g., expandable component 100) will result in the expandable component having a long dimension (e.g., length L) aligned with a central axis of the lumen in which the expandable component is positioned.

In the embodiment illustrated in FIG. 1, because hinge 110 is much narrower than NCS 105 and NCS 106 (x2<<x1 and x2<<x3, respectively), NCS 105 and NCS 106 may bend towards or away from each other in the x-y plane around hinge 110 as indicated by the arrows labeled A and B, which can allow expandable component 100 to accommodate an uneven lumen surface and/or inconsistent lumen diameter.

Referring to FIG. 2, an expandable component 200 includes an NCS 205 and an NCS 206 separated by a hinge 210. Expandable component 200 is similar in this view to expandable component 100 as shown in FIG. 1. A dimension x4 of NCS 205 is greater than a dimension x6 of NCS 206, and both x4 and x6 are greater than a dimension x5 of hinge 210; and a dimension y3 of NCS 205 is greater than a dimension y4 of NCS 206. However, x5 as shown in FIG. 2 is approximately three times greater than x2 as shown in FIG. 1; accordingly, NCS 205 and NCS 206 may have a capability to bend towards or away from each other in the x-y plane that is less than the capability of NCS 105 and NCS 106 to bend towards or away from each other in the x-y plane, while still maintaining some flexibility to allow expandable component 200 to accommodate an uneven lumen surface and/or inconsistent lumen diameter.

As seen by comparing FIG. 1 and FIG. 2, a dimension of a hinge (e.g., hinge 110 or hinge 210) can be designed to have a desired width. Width and other dimensions of an expandable component can be designed, for example, to be suitable for a particular application, to minimize an amount of material used to structure the expandable component, to reduce manufacturing costs, or to increase manufacturing speed.

Referring to FIG. 3, an expandable component 300 includes an NCS 305 and an NCS 306 separated by a hinge 310. A dimension x7 of NCS 305 is greater than a dimension x9 of NCS 306, and both x7 and x9 are greater than a dimension x8 of hinge 310. In this embodiment, a dimension y5 of NCS 305 is approximately equal to a dimension y6 of NCS 306, illustrating another variation of dimensions.

Referring to FIG. 4, an expandable component 400 includes an NCS 405 and an NCS 406 separated by a hinge 410. In this embodiment, a dimension x10 of NCS 405 is less than a dimension x12 of NCS 406, x10 and x12 are both somewhat greater than a dimension x11 of hinge 410, and a dimension y7 of NCS 405 is approximately two times greater than a dimension y8 of NCS 406, illustrating another variation of dimensions.

Other comparative dimensions of NCSs and hinges are within the scope of the present disclosure. For example, a dimension of a hinge in an x-y plane may be greater than a dimension of an NCS in that plane. Further, dimensions may vary in the z-direction within a particular section (NCS or hinge).

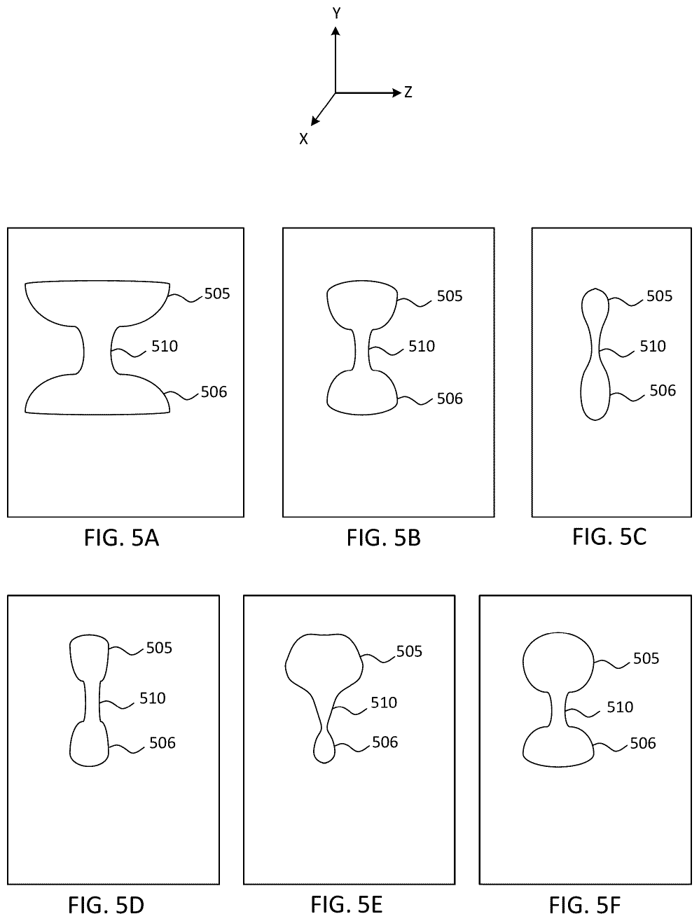

FIGS. 5A-5F illustrate examples of embodiments of expandable components, such as how one or more of expandable components 100, 200, 300, or 400 of FIGS. 1, 2, 3, 4 respectively might look as viewed when rotated (here, rotated into a y-z plane). For convenience with respect to FIGS. 5A-5F, non-compliant sections are referenced as NCS 505, 506 and compliant sections are referenced as hinge 510.

NCS 505 and NCS 506 may bend towards or away from each other in the y-z plane around hinge 510, which can allow the expandable component to accommodate a variety of lumen diameters and shapes.

As can be seen in FIGS. 5A-5F, a shape of an expandable component can vary in a rotated (e.g., side) view, as was the case for the unrotated (e.g., front) view. For example, one (or both) of NCS 505, 506 may have a surface that is substantially flat, somewhat rounded, quite rounded, or other curvature.

It will be apparent from the discussions above that each of the various sections (each NCS and each hinge) may be designed to have desired absolute dimensions as well as desired dimensions comparative to other sections. For example, a hinge may have a width in an x-y plane that is equal to or greater than a width of a particular NCS in the same x-y plane while having a width in a y-z plane that is substantially less than a width of that particular NCS in that y-z plane.

Viewing FIGS. 1-4 and FIGS. 5A-5F with respect to some examples of embodiments of expandable components, it will be apparent that there may be a broad variety of designs for expandable components having two non-compliant sections (NCS) and one compliant section (hinge) in accordance with the present disclosure. In various embodiments, expandable components in accordance with the present disclosure more generally have one or more NCS and one or more hinge.

An expandable component of a self-sizing device may be structured to have one or more sections in each of two or more planes. For example, expandable component 100 shown in a first x-y plane in FIG. 1 may be structured to have additional NCS and/or hinge sections in a second x-y plane such that, as viewed in a y-z plane, expandable component 100 forms a Y-shape, an X-shape, or other multi-pronged shape. A prong may include one or more NCS or no NCS, and/or one or more hinge or no hinge.

An expandable component may include a weakening feature to make it more compliant in a hinging area.

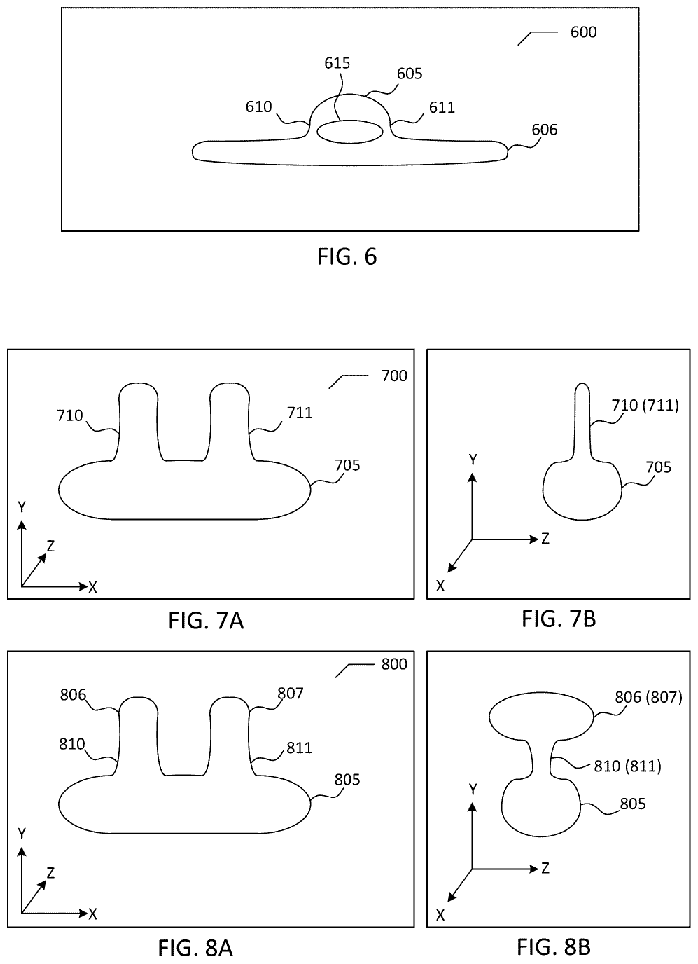

FIG. 6 illustrates an expandable component 600 having two NCSs in the view shown, an NCS 605 and an NCS 606. Expandable component 600 defines an opening 615 illustrated as an oval in this embodiment. Said in another way, expandable component 600 includes a weakening feature shown as opening 615. The omission of material in opening 615 leaves two hinges, a hinge 610 and a hinge 611. Hinges 610 and 611 as fully extended each have at least one design dimension (width, length, and/or circumference) that is substantially smaller than a corresponding design dimension of NCS 605 and NCS 606, which provides for an increase in compliance of hinges 610, 611 relative to NCS 605, 606. Expandable component 600 will tend to bend in this embodiment about hinges 610, 611. Hinges 610, 611 may have similar or different dimensions. When rotated to a different view, expandable component 600 may have a variety of shapes, including the shapes illustrated in FIGS. 5A-5F.

FIG. 7A and FIG. 7B illustrate an expandable component 700 having a single NCS 705 and two hinges, a hinge 710 and a hinge 711. Expandable component 700 is shown in an x-y plane in FIG. 7A, and shown in a y-z plane in FIG. 7B such that hinge 711 is hidden behind hinge 710. In this embodiment, hinges 710, 711 themselves operate to exert pressure against an inner surface of the lumen, while also providing compliance.

FIG. 8A and FIG. 8B illustrate an expandable component 800 having three NCSs, an NCS 805, an NCS 806, and an NCS 807, and two hinges, a hinge 810 and a hinge 811. Expandable component 800 is shown in an x-y plane in FIG. 8A, and shown in a y-z plane in FIG. 8B such that hinge 811 is hidden behind hinge 810 and NCS 807 is hidden behind NCS 806.

As can be seen from FIG. 7A and FIG. 8A, expandable component 700 and expandable component 800 look similar in the x-y planes shown. However, when rotated 90 degrees (e.g., as shown in FIG. 7B and FIG. 8B, respectively, shown in y-z planes), it is seen that NCS 806 and NCS 807 (FIG. 8B) expand to have a dimension in the z-direction that is substantially greater than the corresponding dimension of hinge 710 and 711 (FIG. 7B).

The expandable component may be disposed within a capsule.

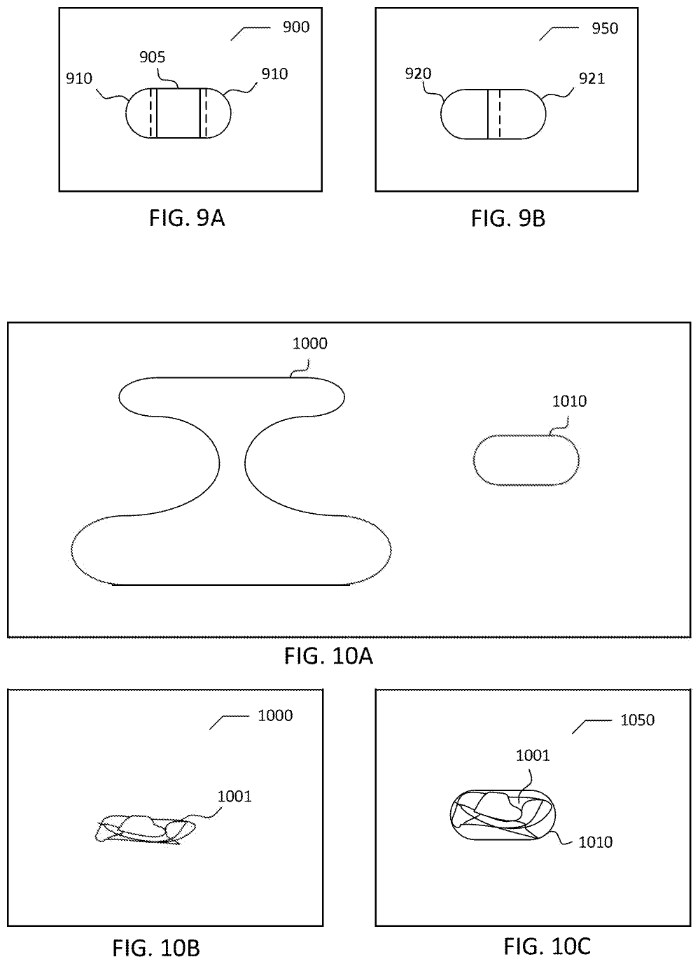

FIG. 9A and FIG. 9B illustrate examples of embodiments of capsules. In FIG. 9A, a capsule 900 includes a cylindrical body 905 and two end caps 910 which are fitted over or into body 905. At least one of the three components of capsule 900 (cylindrical body 905, a first end cap 910, and/or a second end cap 910) is degradable. End caps 910 may be adhered to or press fitted over or in body 905 to retain end caps 910 in position. In FIG. 9B, a capsule 950 includes a portion 920 and a portion 921 which are fitted together. At least one of portions 920, 921 is degradable. Portions 920, 921 may be adhered or press fitted together. Examples of capsule sizes include 000, 00, and 0 capsule sizes. Capsule shapes other than as illustrated in FIGS. 9A, 9B may also be used, such as spherical or ovoid shapes, or irregular shapes, or shapes that are symmetric about a first axis and asymmetric about a second axis perpendicular to the first axis (e.g., egg-shaped), or shapes with at least one flattened side.

In some embodiments, the self-sizing device includes a degradable coating over the expandable component and/or over the capsule (when a capsule is included).

In some embodiments, a coating is applied directly onto an expandable component to complete manufacture of a self-sizing device in lieu of disposing the expandable component within a capsule shell. The coating may completely cover and seal the expandable component, or may cover a portion of the expandable component. For example, the coating may degrade under certain conditions to expose one or more degradable components of the self-sizing balloon device. In turn, the one or more degradable components degrade after being exposed, such as to initiate expansion of the expandable component, or, in an embodiment in which a gas is used to inflate the expandable component, to release the gas and thus deflate the expandable component after delivery of a FVIII composition.

The coating over the expandable device and/or the capsule may be, or may include, an opaque material (e.g., to keep the contents from view), a colored material (e.g., to provide color coding for identifying a FVIII composition contained in the self-sizing device), and/or a material including a noticeable flavor or smell (e.g., undesirable flavor or smell to discourage interest, or desirable flavor or smell to encourage interest), each of which dissolves or degrades sufficiently to allow the self-sizing device to deliver a FVIII composition at the target delivery site.

All, or portions of, the coating and/or the capsule may be designed to degrade after a certain length of time under the conditions expected at a target site, for example after a design period of time after placement, or in the presence of a particular chemical, or under certain values or ranges of pH, temperature, or pressure exerted against the capsule, or a combination of the foregoing. In an embodiment, the capsule is designed to break apart upon an external trigger, for example a trigger wirelessly sent which causes a mechanism within the capsule to rip or break the capsule.

Some embodiments of a self-sizing device according to the present disclosure may be used to deliver a therapeutic FVIII composition into a GI lumen wall via ingestion. The self-sizing device may include a capsule. The self-sizing device may include a coating on the capsule and/or on the expandable component (e.g., a coating as described above), designed to degrade at a particular location within the GI tract, such as within the stomach or the intestine. In an embodiment, all or portions of the capsule and/or the coating are designed to degrade in the presence of a particular chemical, or after a certain length of time under the conditions expected at a target site within the GI tract (e.g., under certain values or ranges of pH, temperature, pressure exerted against the capsule, or a combination of the foregoing). In an embodiment, the coating includes a material to improve swallowability, such as guar gum or xanthan gum.

FIGS. 10A-10C illustrate an example of assembling a self-sizing device 1050, which is a swallowable device as described herein. FIG. 10A illustrates an embodiment of an expandable component 1000 and an embodiment of a capsule shell 1010. Outer dimensions of expandable component 1000 may be several times larger than inner dimensions of capsule 1000. For example, a longest dimension of an embodiment of expandable component 1000 may be two to five times greater than an inner length of capsule shell 1010. Other ratios of outer dimensions of an expandable component to inner dimensions of a capsule are within the scope of the present disclosure. FIG. 108 illustrates expandable component 1000 folded and/or rolled into an arrangement 1001 that is smaller than inner dimensions of capsule shell 1010. As illustrated, arrangement 1001 is folded and/or rolled in a complex arrangement in this embodiment, origami style. FIG. 10C illustrates arrangement 1001 disposed into capsule 1010, and capsule shell 1010 assembled together (e.g., as in FIG. 9A in which end caps 910 are fitted over or into body 905, or as in FIG. 98 in which portions 920, 921 are fitted together). Device 1050 includes expandable component 1000 (in arrangement 1001) and capsule shell 1010, and may include other components not shown such as an expansion module, a delivery component, one or more coatings, and a deflation valve.

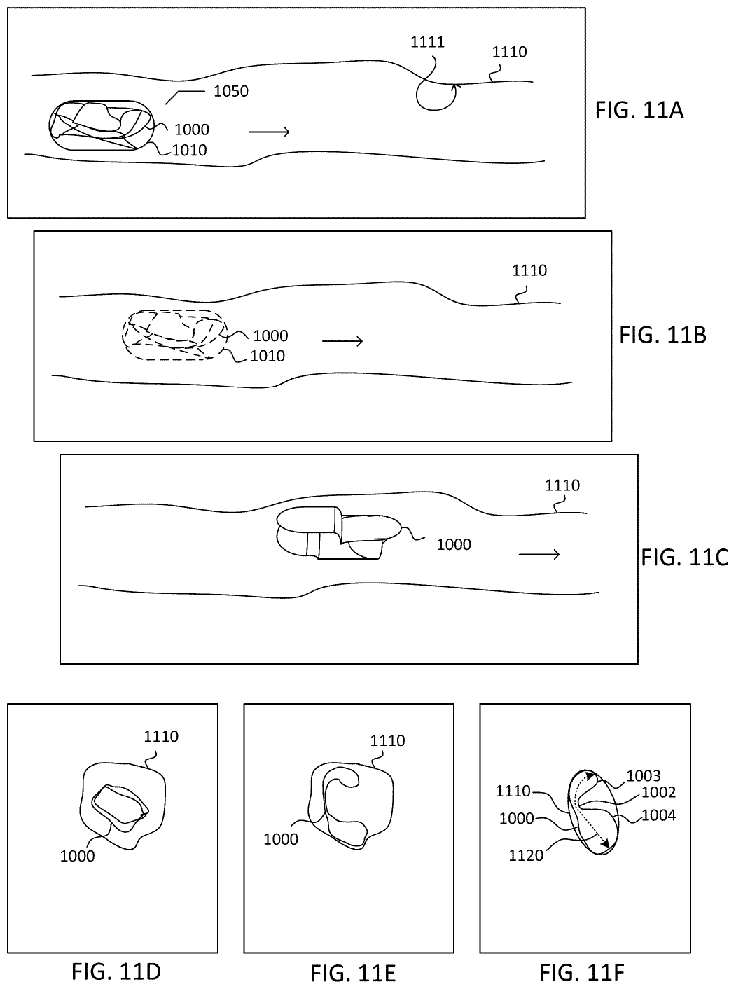

FIGS. 11A-11F illustrate an example of how device 1050 may be provided to a target delivery site in the GI tract and position expandable component 1000 to deliver a therapeutic FVIII composition into a lumen wall 1111 of a GI lumen 1110. Referring back to FIGS. 11A-11C, device 1050 is shown from a view approximately perpendicular to a direction of travel of device 1050 (indicated by an arrow), whereas device 1050 in FIGS. 11D-11F is shown as if looking into lumen 1110 (e.g., approximately 90 degrees from the view of FIGS. 11A-11C).

FIG. 11A illustrates device 1050 within lumen 1110 shown in a relaxed state (e.g., between peristaltic contractions). In this embodiment, an outer diameter of device 1050 is less than an inner diameter of lumen 1110 at this point in the GI tract. Device 1050 traverses lumen 1110, for example through operation of peristalsis, fluid dynamics, and/or gravity.

FIG. 11B illustrates that, after reaching the designed target site or after encountering conditions (e.g., pH) representative of the designed target site, capsule shell 1010 degrades (or is triggered to break open or degrade) as device 1050 continues to traverse lumen 1110. For example with respect to delivery within the intestine, a target site may be in a general region (e.g., small intestine), a more specific region (e.g., jejunum), or a specific tissue area (e.g., a tissue area that has been marked).

FIG. 11C illustrates that, subsequent to (or concurrent with) capsule shell 1010 degrading, expandable component 1000 begins to unfurl from its folded and/or rolled arrangement.

FIG. 11D illustrates expandable component 1000 mostly unfurled, and FIG. 11E illustrates expandable component 1110 as it is expanded (e.g., by an expansion module).

FIG. 11F illustrates expandable component 1000 after expansion. In this embodiment, expandable component 1000 would have been able to expand if not constrained by lumen 1110 until a reference line 1120 was approximately a straight line. However, lumen 1110 exerts force against expandable component 1000 which causes expandable component 1000 to bend at a hinge 1002. An NCS 1003 and an NCS 1004 of expandable component 1000 then press against the lumen wall of lumen 1110 (e.g., at opposing sides of the lumen) and position expandable component 1000 to deliver a therapeutic FVIII composition at or into the lumen wall. Hinge 1002 may also press against the lumen wall. In an embodiment, peristalsis may move expandable component 1000 in its expanded state through lumen 1110. In an embodiment, expandable component 1000 may have sufficient internal pressure (combined with surface tension) to firmly hold expandable component 1000 in approximately a same position even during peristalsis. In an embodiment, expandable component 1000 includes a surface roughness or surface protrusions to assist in maintaining a position of expandable component 1000.

Expansion of an expandable component may be accomplished by any suitable expansion module. In an embodiment, a spring mechanism is released from a compressed state to deploy a filler piece of firm or soft material to push outwards and thus expand the expandable component. In an embodiment, two or more reactants are mixed together to form a gas and thus expand the expandable component. In an embodiment, a material is rapidly combusted to generate a gas and thus expand the expandable component. Other expansion modules are within the scope of the present disclosure.

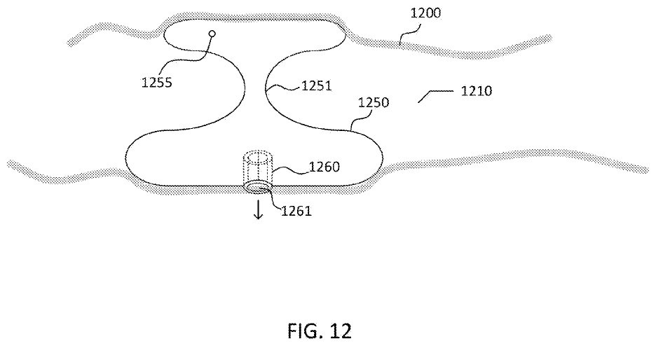

FIG. 12 illustrates a lumen 1200 in which a self-sizing device 1210 including an expandable component 1250 is positioned. In this embodiment, the force of lumen 1200 against expandable component 1250 is not sufficient to substantially cause a hinge 1251 of expandable component 1250 to bend, so expandable component 1250 is in an approximately fully-extended state. Said another way, a bending of hinge 1251 of expandable component 1250 is negligible at this position in this lumen 1200.

Expandable component 1250 may be expanded by generation of gas, and may include a valve 1255 to release the gas from within expandable component 1250 into lumen 1200 to deflate expandable component 1250. Valve 1255 may include one or more components. In an embodiment, valve 1255 is a pinch valve that degrades in the presence of fluid (e.g., biological matter) to reveal a port or an opening defined by expandable component 1250. In an embodiment, valve 1255 is a piece of material or a coating over a port or an opening in expandable component 1250, and the piece of material or coating is designed to degrade a period of time after expandable component 1250 delivers a therapeutic FVIII composition. In an embodiment, valve 1255 is opened manually.

The therapeutic composition of FVIII (or multiple therapeutic composition) can be stored within expandable component 1250 in any constitution (e.g., as a solid, fluid, slurry, or powder). The therapeutic composition(s) can be delivered in any constitution (e.g., as a solid, fluid, slurry, or powder). In some embodiments, two or more components of the therapeutic composition are mixed within expandable component 1250 prior to delivery. In an embodiment, expandable component 1250 includes a portal such as portal 1260 defining an opening 1261 through which the therapeutic composition is delivered. In embodiments that include a tissue penetrating member, the tissue penetrating member may be delivered through the portal.

The therapeutic FVIII composition is actively released such that the therapeutic FVI II composition is forcibly expelled from expandable component 1250 and through lumen 1200 and beyond (e.g., through the mucosa, submucosa, muscularis, serosa, and an outer wall of lumen 1200, and into the peritoneal cavity); in such an embodiment, the therapeutic FVIII composition may be released through one or more portals such as portal 1260.

The therapeutic composition of FVIII may be forcibly expelled from expandable component 1250 by a delivery mechanism. For example, with respect to a solid composition (e.g., tablet, pellet, or pointed form), the therapeutic FVIII composition may be expelled by way of a spring mechanism that is released to quickly eject the solid composition out of expandable component 1250, or by way of a piston mechanism in which the piston is moved by a spring mechanism or a gas expansion to push the solid composition quickly out of expandable component 1250.

In some embodiments, a surface of expandable component 1250 through which the therapeutic FVIII composition is delivered contacts the wall of lumen 1200 along a stretch of approximately 5 mm to 20 mm.

In some embodiments, the therapeutic FVIII composition is delivered from expandable component 1250 from multiple surfaces. Such embodiments may include more than one tissue penetrating member that is formed from or contains a FVIII composition.

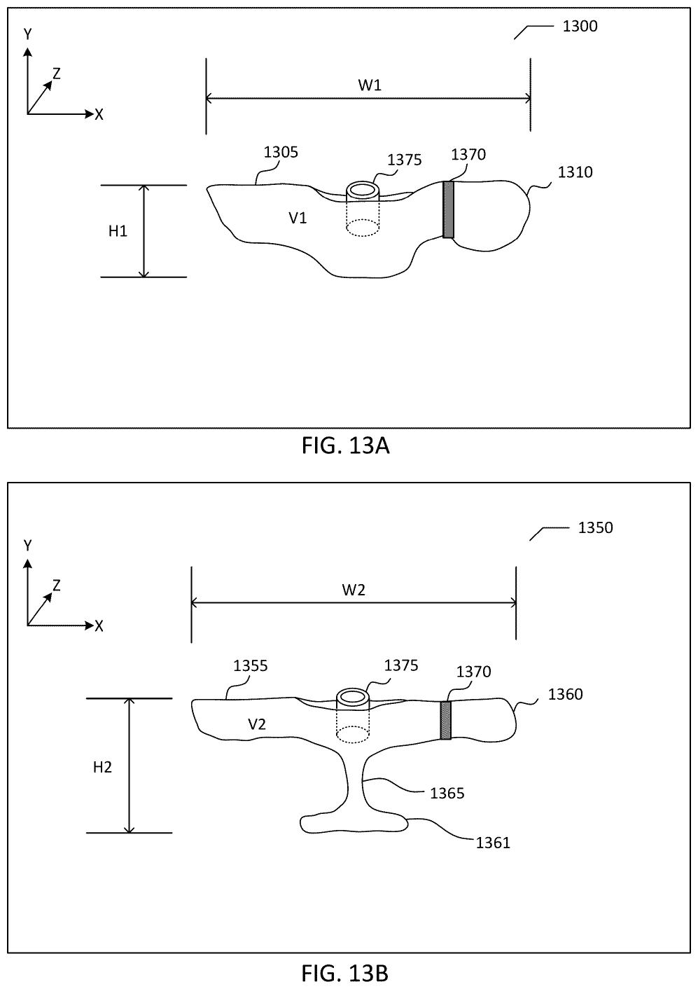

FIG. 13A illustrates an embodiment of a self-sizing device 1300 including an expandable component 1305 which includes one NCS 1310 and no hinge. FIG. 13B illustrates an embodiment of a self-sizing device 1350 including an expandable component 1355 which includes two NCSs, an NCS 1360 and an NCS 1361, and a hinge 1365 between. With respect to these embodiments, a width W1 of expandable component 1305 is similar to a width W2 of expandable component 1355, whereas a height H1 of expandable component 1305 is less than a height H2 of expandable component 1355.

Multiple devices 1300 (FIG. 13A) structured according to a same design will have approximately a same maximum circumference of NCS 1310 (in a y-z plane) when fully inflated in a lumen due to the non-conformance of NCS 1310, such that expandable component 1305 does not significantly adapt to a variety of different internal lumen circumferences. Different sizes of device 1300 may therefore be appropriate for different animal species and/or different subjects within a species. In contrast, multiple devices 1350 (FIG. 13B) structured according to a same design will likely not have a same maximum circumference (in a y-z plane) when fully inflated in a lumen because each device 1350 will adjust to the size of the lumen in which it is inflated due to the flexibility (non-conformance, bending) of hinge 1365 even when inflated. Maximum circumference with respect to device 1350 within the lumen refers to a maximum circumference of the shape that device 1350 takes within the lumen (e.g., a partially-extended shape or a fully-extended shape).

Device designs similar to the illustrations of devices 1300, 1350 have been prepared. For convenience of reference, the identifying numerals used in FIG. 13A and FIG. 13B are used to describe these trial devices in the following discussion. For these trial devices, an expansion module included two reactants (sodium bicarbonate and citric acid) and a biodegradable pinch valve 1370 that separated the two reactants. Valve 1370 was designed to degrade and allow the reactants to mix to generate carbon dioxide to inflate expandable component 1305 or expandable component 1355.

For various trials, trial devices were constructed according to one of three example designs: Device A, Device B, and Device C. Device A and Device B were similar to device 1300 (FIG. 13A), and Device C was similar to device 1350 (FIG. 13B).

Device A had a maximum circumference (in a y-z plane) of approximately 65 mm when fully inflated and unconstrained. Device B had a maximum circumference (in a y-z plane) of approximately 68 mm when fully inflated and unconstrained. For clarity, Device A and Device B had a similar shape and similar width (a width of Device A and a width of Device B were approximately equal to W1), but Device B was taller than Device A (e.g., a height of Device A was equal to H1 and a height of Device B was greater than H1). Device C had a maximum circumference (in a y-z plane) of approximately 80 mm when fully inflated and unconstrained.

As expanded, an interior volume V1 of expandable component 1305 of Device A was approximately 8 cubic centimeters (cc), whereas an interior volume V2 of expandable component 1355 of Device C was approximately 4 cc. The reduced volume of expandable component 1355 was due in part to NCS 1360 and NCS 1361 of device 1350 each and collectively having a smaller circumference (in a y-z plane) than NCS 1310 of device 1300. The reduced volume of expandable component 1355 of Device C was achieved even though the overall height H2 of expandable component 1355 was greater than the overall height H1 of expandable component 1305 of Device A. The greater height of expandable component 1355 allowed for deployment in larger-diameter lumens. The smaller volume of expandable component 1355 allowed for a reduction in an amount of reactants needed as compared to an amount of reactants needed in expandable component 1305 to achieve a same internal pressure.

In some embodiments, a delivery mechanism 1375 may include a piston that delivers a therapeutic FVIII composition by pressure of the carbon dioxide against the piston, causing the piston to rapidly move and eject a solid form of the FVIII composition into a wall of the lumen. In embodiments that include a tissue penetrating member, the tissue penetrating member may be delivered via the piston.

In some embodiments, increasing a circumference of the expandable component (Device A compared to Device B) may result in a better delivery rate across a set of subjects. However, the delivery rate in a particular human may be affected by a maximum circumference (in the y-z plane) of a device with no hinge, such that, for some therapeutic treatments, each human might be tested with multiple devices (each with different maximum circumference) prior to or at the beginning of the therapeutic treatment, to identify which device size was the most appropriate to use for that human.

In some embodiments, a single size and design of an expandable component including a hinge (e.g., Device C) could be used successfully across a high percentage of subjects. For devices structured similarly to Device C, it was shown that delivery rate was consistent whether the subjects were fed, or were fasted for several hours prior to ingestion of the device.

Adding a hinge to an expandable component allows for the use of a consistently sized device. A single design of a hinged expandable component in accordance with the present disclosure can deliver a composition of FVIII into a lumen with a high rate of delivery over a selected range of circumferences of an inner wall of the lumen. In an embodiment, a device is designed for delivery within the small intestine in humans, and the selected range is about 10 mm to about 100 mm, or about 20 mm to about 80 mm. In some embodiments, a self-sizing device suitable for use at any of various locations along the GI tract (e.g., stomach, small intestine, large intestine, colon) over a variety of animal species is designed to have a selected range of about 5 mm to about 500 mm. Other ranges may be selected, and different ranges may be selected for different types of lumens within the body, or for other types of lumens.

In some embodiments, an expandable component is designed with at least one hinge visible from a first viewing angle and multiple hinges visible from a second viewing angle rotated from the first viewing angle, such that each of three or more sections (NCS and/or hinge sections) of the expandable component contacts a lumen in which the expandable component is disposed. This structure may allow compliance of multiple hinges to adapt the expandable component to a wider selected range of lumen circumferences.

Another benefit of the design concept using a hinge is that a total surface area of the expandable component may be reduced as compared to an expandable component not using a hinge, while still allowing for a single self-sizing device design to be used for applications in a wide variety of lumens, in a wide variety of animal species, and in a wide variety of subjects within a species. Accordingly, a reduced amount of material may be needed; this reduction in material, along with a reduced amount of reactants, may result in cost savings.

In some embodiments, an expandable component is structured using one type of material throughout. In an embodiment, an expandable component is structured using two or more different materials. In some embodiments, a level of compliance of an expandable component may be adjusted based on a thickness of material at certain areas of the expandable component. In some embodiments, a level of compliance of an expandable component may be adjusted by adding layers of a same material or different materials at certain areas of the expandable component. Examples of materials which may be used to structure an expandable component include polyester, polyethylene terephthalate (PET), high-density polyethylene (HDPE), a cross linked polymer, silicone, polyurethane, or other elastomer or polymer.

In some embodiments, a delivery mechanism (e.g., delivery mechanism 1375) includes an advancement device (e.g., a piston or rod) directly or otherwise operably coupled to the payload to exert a force on a surface of the payload to advance it into a lumen wall. The delivery mechanism may be in operative contact with or otherwise closely positioned adjacent to the lumen wall when the expandable component is expanded, such that the payload is ejected directly into the lumen wall with a minimal gap or no gap between a surface of the delivery mechanism and the lumen wall, which may improve a delivery rate of the self-sizing device. In embodiments that include a tissue penetrating member, the tissue penetrating member may be delivered via the advancement device.

In some embodiments, a self-sizing device includes a payload detachably coupled to a piston so that after advancement of the payload to the lumen wall, the payload detaches from the piston. In embodiments that include a tissue penetrating member, the payload may be formed into or contained within the tissue penetrating member and detachably coupled to a piston.

Specific materials can be chosen to confer desired structural and material properties to the payload (e.g., column strength for insertion into the lumen wall, or porosity and/or hydrophilicity for controlling disintegration of the payload and thus the release of a therapeutic FVIII composition). For example, certain material may be more suitable when the payload is formed as a tissue penetrating member, whereas other material may be more suitable when the payload is contained within a tissue penetrating member. In an embodiment, a tip of the payload includes or is coated with a degradable material such as sucrose, maltose, or other sugar, to increase hardness and tissue piercing properties of the tip. Once positioned within a lumen wall, the payload may be degraded by interstitial fluids within tissue so that the therapeutic FVIII composition dissolves and is absorbed into the blood stream. Properties of a payload such as size, shape, and chemical composition can be selected to allow for dissolution and absorption of a drug in a matter of seconds, minutes, or hours. Rates of dissolution can be controlled through various excipients such as disintegrants (e.g., starch, sodium starch glycolate, or a cross-linked polymer such as carboxymethyl cellulose). The choice of disintegrants may be specifically adjusted for the environment within a lumen wall (e.g., blood flow and average number of peristaltic contractions).

A payload can be fabricated entirely from a FVIII composition, or can define a cavity that includes a FVIII composition. A self-sizing device can include and deliver one or more payloads, each of which can contain the same or a different FVIII composition from another of the payloads. Each payload can have different properties; for example, two payloads may be designed to deliver respective compositions concurrently (e.g., two instances of one composition, or one instance each of two distinct compositions), or may be designed to deliver respective compositions at different times (e.g., to provide a subsequent dose of a same FVIII composition or to provide a different FVIII composition subsequently).

While specific embodiments have been described and illustrated, it should be understood that components or characteristics of one embodiment can be combined or substituted with one or more components or characteristics from other embodiments. Further, for any positive recitation of a component, characteristic, constituent, feature, step or the like, the present disclosure specifically contemplates the exclusion of that component, value, characteristic, constituent, feature, step or the like. It also should be understood that illustrations may not necessarily be drawn to scale. There can be distinctions between the artistic renditions in the present disclosure and the actual apparatus, due to variables in manufacturing processes and such. There can be other embodiments of the present disclosure which are not specifically illustrated. Thus, the specification and drawings are to be regarded as illustrative rather than restrictive.

The following examples are given to illustrate the present invention. It should be understood, however, that the invention is not to be limited to the specific conditions or details described in these examples.

### EXAMPLES

### Example 1— Preparation of Factor VIII Capsule Devices

A solution of PEGylated recombinant human FVIII (ADYNOVATE®) was lyophilized to obtain a powder. Microtablets were manufactured using the lyophilized powder under aseptic conditions, targeting a dose of 3,000 IU/microtablet. FVIII activity was assessed using a standard chromogenic assay at each processing step to ensure that the biological activity remained high and was not substantially degraded by reformulation and microtabletting. The FVIII microtablets were incorporated into devices within a capsule as described above with reference to FIG. 18, with a payload in the form of a hollow microneedle containing a single microtablet, and the capsule device containing a single payload. The FVIII-loaded capsule devices were shipped interstate and stored at 2-8° C. for one week. FVIII activity of the capsule devices was determined on the day of manufacture and after one week. The FVIII activity was substantially the same after one week as it was at manufacture.

### Example 2—Orally Delivered Factor VIII Normalizes Hemostasis in Hemophilia a Canines

A first hemophilia A canine received a direct intraperitoneal (IP) injection of FVIII. The dose delivered by the IP injection was about 150 IU/kg of PEGylated FVIII (ADYNOVATE®). The IP-injected FVIII provided comparison (reference) data to a test of capsule-delivered FVIII by way of a swallowable capsule device and a method as disclosed herein; the test is described next.

Orally ingestible capsule devices were prepared as in Example 1, with microtablets composed of PEGylated human recombinant FVIII (ADYNOVATE®; approximately 2,650 IU). The capsules were shipped to a study site one week prior to commencing the study and stored at 2-8° C. A capsule was administered orally to a second, awake hemophilia A canine. The canine was 16.8 kg; accordingly, the dose was approximately 158 IU/kg, comparable to the approximately 150 IU/kg used for the IP-injected FVIII described above. To track the transit of the capsule across the GI tract as well as to determine the time of deployment (T=0) for initiating serial blood sample collection, abdominal X-ray imaging was performed every 30 minutes after ingestion of the capsule while the capsule was in the stomach, and then every 5-15 minutes once the capsule was in the small intestine. Gastric emptying time (GET) and intestinal deployment time (IDT) were tracked and recorded. The GET was measured from the time of ingestion of the capsule to the time of confirmed exit out of the stomach and into the small intestine. IDT was measured from the time of confirmed entry of the capsule into the small intestine to the time that the microneedle was delivered into the intestinal wall and thus to the peritoneal cavity. The GET was 60 minutes and the IDT was 50 minutes.

For both the IP-injected dose and the swallowable capsule device-delivered dose, serial blood samples (10 ml) were collected into sodium citrate treated tubes over 7 days to assess changes in pharmacokinetic (PK) parameters by measuring FVIII levels and activity, and changes in pharmacodynamic (PD) parameters by evaluating whole blood clotting time (WBCT), thromboelastography (TEG), and activated partial thromboplastin time (aPTT). PK assessment was performed with the COAMATIC® FVIII assay by Chromogenix.

Table 1 summarizes and explains the PK and PD tests that were performed for this study.

TABLE 1
Study Endpoints

| Purpose | Test | Test Measure |
|---|---|---|
| Pharmacokinetics | FVIII Antigen Assay | Quantifies amount of FVIII |
| | | present in plasma by ELISA |
| | FVIII Activity Assay | Measures FVIII activity |
| | | levels |
| Pharmacodynamics | Activated Partial | Evaluates integrity |
| | Thromboplastin | of intrinsic |
| | Time (aPTT) | coagulation pathway |
| | Whole Blood Clotting | Determines coagulation |
| | Time (WBCT) | time of whole blood |
| | Thromboelastography | Quantifies the ability of |
| | (TEG) | whole blood to clot |

PK and PD parameters from the swallowable capsule device-delivered dose of FVIII were compared to PK and PD parameters from the IP-injected dose of FVIII. For each test, T=0 was the time that the microneedle of the swallowable device was delivered into the intestinal wall and thus to the peritoneal cavity with respect to the capsule-delivered dose, and the time of injection with respect to the IP-injected dose.

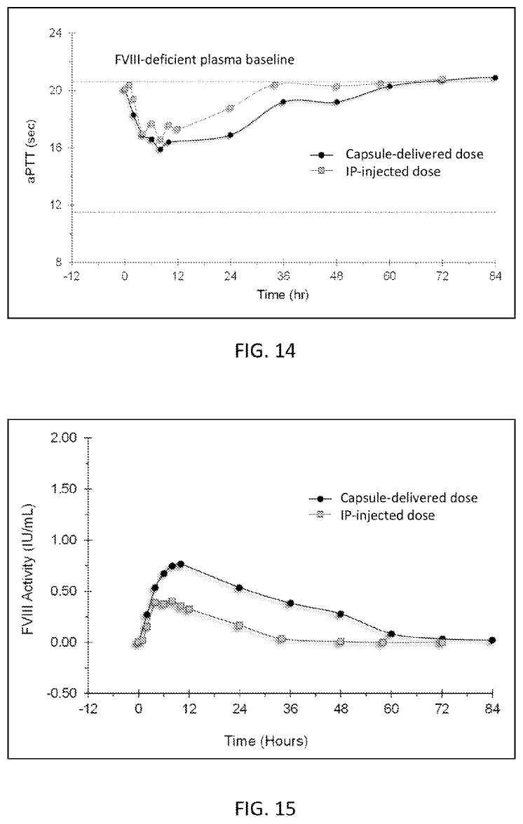

Both the capsule-delivered dose and the IP-injected dose normalized coagulopathy for up to 3 days (FIG. 14) with concomitant changes in plasma FVIII concentrations and activity (FIG. 15). The time-course and pattern of PK and PD changes with capsule-delivered FVIII administration in accordance with the methods and uses disclosed herein resembled that following IP-injected administration of FVIII. The canines remained normal and healthy throughout the study period (except for the pre-existing hemophilia A condition).

Table 2 shows the TEG parameters for the capsule-delivered FVIII dose (the “Capsule” FVIII Route of admin.) and the IP-injected FVIII dose (the “IP” FVIII Route of admin.). The WBCT assays closely corresponded to and supported the results of the TEG data.

TABLE 2
Thromboelastographic parameters after FVIll administration via IP injection and capsule delivery

| TEG Parameter | FVIII Route | Time Point (hr) | | | | | | | |
|---|---|---|---|---|---|---|---|---|---|
| (Ref. Range) | of admin. | PD | 4 | 8 | 12 | 24 | 48 | 72 | 96 |
| Reaction Time | IP | 34.1 | 7.8 | 12.5 | 13 | 12.9 | 31.7 | >60 | — |
| (5-10 min) | Capsule | 39.7 | 9.8 | 7.0 | 6.7 | 9.3 | 16.5 | 14.5 | 26 |
| Kinetic Value | IP | 13.8 | 2.2 | 3.1 | 2.9 | 3.6 | 11.2 | — | — |
| (1-3 min) | Capsule | 14.2 | 1.8 | 1.3 | 1.5 | 1.9 | 2.7 | 3.0 | 6.6 |
| Angle | IP | 16.4 | 60.5 | 50.1 | 49.5 | 44.4 | 18.9 | — | — |
| (53-72°) | Capsule | 11 | 67 | 69.9 | 67.9 | 63.8 | 54.6 | 53.5 | 27.3 |
| Maximal Amplitude | IP | 29.5 | 58.8 | 49.6 | 52.9 | 52.2 | 43.5 | — | — |
| (50-70 mm) | Capsule | 39.3 | 57.9 | 59.5 | 66.1 | 52.4 | 51.0 | 56.0 | 56.8 |

As can be seen from the data presented in the discussion above and in the figures, a single capsule-delivered dose of FVIII administered in accordance with the methods and uses disclosed herein restored markers of hemostasis in a canine model of hemophilia A for at least 3 days. The data and the figures also show that the IP injection also restored hemostasis in the hemophilia A dog in a manner that showed a striking similarity to the onset and time course of changes in the PK and PD parameters.

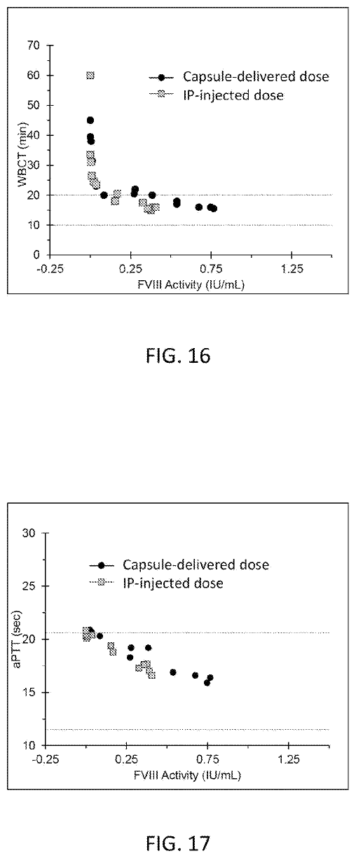

PK-PD analysis indicates that the FVIII payload was successfully delivered into the peritoneal space, as shown by the striking similarity of the data for the IP-injected dose versus the capsule-delivered dose, such as WBCT plotted against FVIII Activity (FIG. 16) and aPTT plotted against FVIII Activity (FIG. 17).

These data underscore the surprising advantages of the methods disclosed herein, especially when compared to the current standard of care that requires frequent intravenous injections of FVIII.

1. A method of treating hemophilia A, comprising orally administering, to a subject with hemophilia A, a swallowable device containing a payload formed from, or containing, a composition comprising Factor VIII (FVIII), wherein the device is structured to deliver the FVIII into a peritoneal cavity of the subject, wherein the administering is at a frequency selected from twice every day, once every day, once every two days, and once every three days.

2. A method of treating hemophilia A, comprising orally administering, to a subject with hemophilia A, a swallowable device containing a payload formed from, or containing, a composition comprising Factor VIII (FVIII), wherein the device is structured to deliver the FVIII into a peritoneal cavity of the subject, wherein the device contains a dose of FVIII of from about 30 IU/kg to about 300 IU/kg.

3. A method of treating hemophilia A, comprising orally administering, to a subject with hemophilia A, a swallowable device containing a payload formed from, or containing, a composition comprising Factor VIII (FVIII), wherein the device is structured to deliver the FVIII into a peritoneal cavity of the subject, wherein the method is effective to protect the subject from breakthrough bleeding for at least 72 hours and up to 120 hours.

4. The method of claim 3, wherein the breakthrough bleeding is spontaneous bleeding.

5. The method of claim 2, wherein the device is administered daily.

6. The method of claim 1, wherein the device contains a dose of FVIII of from about 1,000 IU to about 12,000 IU.

7. The method of claim 6, wherein the device contains a dose of FVIII of 3,000 IU, 6,000 IU, or 9,000 IU.

8. The method of claim 1, wherein the composition comprising FVIII is a dry composition filled in a hollow, biodegradable microneedle constituting the solid tissue penetrating member.

9. The method of claim 1, wherein the FVIII is a long-circulating form of FVIII.

10. The method of claim 1, wherein the FVIII is a recombinant human PEGylated FVIII.

11. The method of claim 2, wherein the method comprises administering the device to the subject at a frequency selected from once every 2 days, once every 3 days, once every 4 days, once every 5 days, once every 6 days, and once every 7 days.

12. The method of claim 1, wherein the dose of FVIII is selected from about 75 IU/kg, about 100 IU/kg, about 125 IU/kg, about 150 IU/kg, about 175 IU/kg, about 200 IU/kg, about 225 IU/kg, about 250 IU/kg, and about 275 IU/kg.

13. The method of claim 1, wherein the dose of FVIII is from about 50 IU/kg to about 250 IU/kg, from about 50 IU/kg to about 200 IU/kg, from about 50 IU/kg to about 150 IU/kg, or from about 50 IU/kg to about 100 IU/kg.

14. The method of claim 1, wherein the method is effective in maintaining one or both of hemostasis and normalized coagulopathy in the subject for a period of time selected from at least 24 hours, at least 36 hours, at least 48 hours, at least 60 hours, at least 72 hours, at least 84 hours, or at least 96 hours, and up to 120 hours.

15. The method of claim 1, wherein the method is effective to protect the subject from one or both of breakthrough bleeding and spontaneous bleeding for a period of time selected from at least 24 hours, at least 36 hours, at least 48 hours, at least 60 hours, at least 72 hours, at least 84 hours, or at least 96 hours, and up to 120 hours.

16. The method of claim 1, wherein the method is effective to maintain FVIII at therapeutic levels in the subject for an equal or longer period of time than intravenous administration of an equal dose of FVIII.

17. The method of claim 1, wherein the payload is in the form of a solid tissue penetrating member structured to penetrate an intestinal wall of the subject and/or be inserted into a peritoneal cavity of the subject after oral ingestion of the swallowable device.

18. The method of claim 1, wherein the device comprises multiple payloads or doses of FVIII.

19. The method of claim 18, wherein the device is structured to deliver one or more payloads or doses into the peritoneal cavity of the subject at different times.

20. The method of claim 18, wherein the device comprises 2, 3, 4, or 5 or more payloads or doses of FVIII.

21. The method of claim 1, wherein the swallowable device is comprised within a capsule.

22-24. (canceled)
